(19) United States
(12) Patent Application Publication
(10) Pub. No.: US 2016/0374565 A1
(43) Pub. Date: Dec. 29, 2016

(54) OBJECT INFORMATION ACQUIRING APPARATUS, OBJECT INFORMATION ACQUIRING METHOD, AND STORAGE MEDIUM

(71) Applicant: CANON KABUSHIKI KAISHA, Tokyo (JP)

(72) Inventor: Takao Nakajima, Kawasaki-shi (JP)

(21) Appl. No.: 15/184,249

(22) Filed: Jun. 16, 2016

(30) Foreign Application Priority Data
Jun. 25, 2015 (JP) 2015-127704

(51) Int. Cl. A61B 5/00; A61B 5/1455

(52) U.S. Cl. CPC A61B 5/0095; A61B 5/14551; A61B 5/7246; A61B 5/7275; A61B 5/742

(57) ABSTRACT

An object information acquiring apparatus is used, which includes: a conversion element receiving an acoustic wave from an object irradiated with light with a plurality of wavelengths and converts the acoustic wave into an electric signal; a memory storing a lookup table or a relational expression that represents a relation between the wavelength of light and a transformation coefficient based on an intensity of an acoustic wave from a standard sample with a known coefficient relating to light absorption and the coefficient for the standard sample; a transformation coefficient acquiring unit that acquires the transformation coefficient from the memory; and a signal processing unit acquiring specific information on the object, using an electric signal and the transformation coefficient.

### BACKGROUND OF THE INVENTION

Field of the Invention

The present invention relates to an object information acquiring apparatus, an object information acquiring method, and a storage medium.

Description of the Related Art

A photoacoustic imaging technology is an imaging technology using light. In the photoacoustic imaging, first, pulsed light emitted from a light source is radiated to an object. The irradiation light propagates and diffuses through the object. The energy of the light is absorbed at a plurality of positions in the object to generate acoustic waves (hereinafter referred to as photoacoustic waves). The photoacoustic waves are received by a transducer, and the received signals are analyzed in a processing apparatus to acquire optical specific values for the inside of the object in the form of image data. Consequently, the distribution of optical specific values in the object is visualized.

In recent years, resolution has been desired to be increased in order to allow finer light absorbers to be imaged using photoacoustic waves. Every effort has been made to develop photoacoustic microscopes that image absorbers such as fine blood vessels near the surface of the object at a high resolution by focusing sound or condensing pulsed light.

An initial sound pressure (P₀) of a photoacoustic wave emitted from an absorber in the object as a result of light absorption is represented by Math. 1.

[Math. 1]

P₀=Γ·μₐ·Φ (1)

In the expression, a Gruneisen coefficient is denoted by Γ and determined by multiplying a coefficient of volume expansion (β) by the square of the speed of sound (c) and dividing the product by a specific heat at constant pressure (Cₚ). The amount of light at a certain position (local area) is denoted by Φ. The amount of light is the amount of light radiated to the absorber and is also referred to as light influence. The coefficient of light absorption at a certain position is denoted by μₐ. The initial sound pressure (P₀) can be calculated using a reception signal (PA signal: photoacoustic signal) output from a probe having received a photoacoustic wave.

Math. 1 indicates that, in theory, the amount of light Φ needs to be acquired in order to determine the absorption coefficient μₐ.

An apparatus in Japanese Patent Application Laid-open No. 2010-088627 measures the shape of a living organism and the distribution of light irradiation and calculates the distribution of the amount of light Φ in the object based on the results of the measurement and an average optical coefficient for the inside of the living organism.

Patent Literature 1: Japanese Patent Application Laid-open No. 2010-088627

### SUMMARY OF THE INVENTION

However, in photoacoustic apparatuses including photoacoustic microscopes, it may be difficult to determine the effects of changes in the intensity of light radiated to the object and of changes in reception signal and to accurately acquire specific information. The present invention has been developed in view of the above-described problems. An object of the present invention is to provide a method of easily acquiring specific information on the object using an apparatus that acquires a photoacoustic wave emitted from the object irradiated with light.

The present invention provides an object information acquiring apparatus comprising:

a transformation coefficient acquiring unit configured to acquire, based on a lookup table or a relational expression that represents a relation between (a) a transformation coefficient for transformation between an intensity of an acoustic wave resulting from radiation of light to a standard sample with a known coefficient relating to light absorption and the coefficient relating to the light absorption of the standard sample and (b) a wavelength of light, the transformation coefficient corresponding to the wavelength of the light; and

a signal processing unit configured to acquire specific information on an object using the transformation coefficient and an electric signal derived from the acoustic wave generated by the object irradiated with light.

The present invention also provides an object information acquiring method comprising:

acquiring a lookup table or a relational expression that represents a relation between (a) a transformation coefficient for transformation between an intensity of an acoustic wave resulting from radiation of light to a standard sample with a known coefficient relating to light absorption and the coefficient relating to the light absorption of the standard sample and (b) a wavelength of light;

acquiring the transformation coefficient corresponding to the wavelength of the light, based on the lookup table or the relational expression; and

acquiring specific information on an object, using the transformation coefficient and an electric signal derived from the acoustic wave generated by the object irradiated with light.

The present invention also provides a non-transitory computer readable storage medium having a program stored therein, the program allowing a computer to execute:

acquiring a lookup table or a relational expression that represents a relation between (a) a transformation coefficient for transformation between an intensity of an acoustic wave resulting from radiation of light to a standard sample with a known coefficient relating to light absorption and the coefficient relating to the light absorption of the standard sample and (b) a wavelength of light;

acquiring the transformation coefficient corresponding to the wavelength of the light, based on the lookup table or the relational expression; and

acquiring specific information on an object, using the transformation coefficient and an electric signal derived from the acoustic wave generated by the object irradiated with light.

The present invention can provide a method of easily acquiring specific information on the object using an apparatus that acquires a photoacoustic wave emitted from the object irradiated with light.

Further features of the present invention will become apparent from the following description of exemplary embodiments with reference to the attached drawings.

### BRIEF DESCRIPTION OF THE DRAWINGS

FIG. 1 is a schematic diagram depicting a general configuration of a photoacoustic apparatus to which Embodiment 1 can be applied;

FIG. 2 is a flowchart illustrating an example of a measurement flow in Embodiment 1;

FIG. 3 is a schematic diagram depicting a general configuration of a photoacoustic apparatus in another form to which Embodiment 1 can be applied;

FIG. 4 is a schematic diagram depicting a general configuration of a photoacoustic apparatus to which Embodiment 2 can be applied;

FIG. 5 is a flowchart illustrating an example of a measurement flow in Embodiment 2;

FIG. 6 is a schematic diagram depicting a general configuration of a photoacoustic apparatus to which Embodiment 3 can be applied;

FIG. 7 is a flowchart illustrating an example of a measurement flow in Embodiment 3;

FIG. 8A and FIG. 8B are diagrams illustrating the concept of creation of a lookup table;

FIG. 9A, FIG. 9B, and FIG. 9C are tables illustrating the effects of a depth that is an object feature amount; and

FIG. 10A, FIG. 10B, and FIG. 10C are tables illustrating the effects of an inclination that is an object feature amount.

### DESCRIPTION OF THE EMBODIMENTS

With reference to the drawings, preferred embodiments of the present invention will be described. However, the dimensions, materials, shapes, and relative arrangements of components described below should be changed as needed, according to a configuration of an apparatus to which the present invention is applied and various conditions. Hence, the scope of the present invention is not intended to be limited to the description below.

The present invention relates to a technique for detecting an acoustic wave propagating from a living organism and generating and acquiring specific information on the object. Hence, the present invention is considered to be an object information acquiring apparatus or a control method for the object information acquiring apparatus, or an object information acquiring method and a signal processing method. The present invention is also considered to be a program that allows an information processing apparatus (computer) including hardware resources such as a CPU and a memory to execute the methods and a storage medium that stores the program.

The object information acquiring apparatus of the present invention includes an apparatus that, in use of a photoacoustic effect, irradiates an object with light (electromagnetic wave) to receive an acoustic wave generated in the object, thus acquiring specific information on the object in the form of image data. The specific information in the present invention is information on specific values (specific-value information) corresponding to respective plurality of positions in the object which information is generated using reception signals resulting from reception of photoacoustic waves.

The object information acquiring apparatus of the present invention includes an apparatus referred to as a photoacoustic microscope that focuses at least one of irradiation light and the photoacoustic wave. Such an apparatus may allow a received photoacoustic wave to be utilized as specific information on the object simply by executing an easy process such as envelope detection on the photoacoustic wave. A photoacoustic microscope apparatus and a photoacoustic tomography apparatus are also simply referred to as photoacoustic apparatuses.

The acoustic wave as used herein is typically an ultrasonic wave and includes an elastic wave referred to as a sound wave or an acoustic wave. An electric signal into which an acoustic wave is converted by a probe or the like is also referred to as an acoustic signal. However, the description of the “ultrasonic wave” or “acoustic wave” as used herein is not intended to limit the wavelength of the elastic wave. An acoustic wave resulting from a photoacoustic effect is referred to as a photoacoustic wave. An electric signal derived from a photoacoustic wave is also referred to as a photoacoustic signal.

The object information acquiring apparatus in embodiments described below can be utilized, for example, for diagnosis of vascular diseases in human beings and animals and for follow-up of chemical treatment.

The specific information acquired in the present invention is a value reflecting the absorptivity of light energy. For example, the specific information includes a source of a photoacoustic wave resulting from light irradiation, an initial sound pressure in the object or a light energy absorption density or coefficient derived from the initial sound pressure, and the concentrations of substances forming a tissue. For the concentrations of substances, determining an oxyhemoglobin concentration and a deoxyhemoglobin concentration allows an oxygen saturation distribution to be calculated. The following can also be determined: a glucose concentration, a collagen concentration, a melanin concentration, and the volume factions of fat and water. A two- or three-dimensional specific-information distribution is also obtained based on specific information for various positions in the object. Distribution data is generated in the form of image data.

Irradiation with a plurality of light beams with different wavelengths allows acquisition of a distribution for the concentrations of substances present in the object. In this case, an absorption coefficient μa for the inside of the object is determined for each wavelength, and the distribution for the concentration of each substance is imaged by using the determined values and wavelength dependence specific to the substance of interest.

In particular, the oxygen saturation of blood can be acquired based on the concentrations of oxyhemoglobin HbO and deoxyhemoglobin Hb. In this case, the oxygen saturation is calculated using light absorption distribution data for each measurement wavelength and absorbance spectra for the oxyhemoglobin HbO and the deoxyhemoglobin Hb. When two wavelengths are used, the oxygen saturation SO₂ is determined by Math. 2.

[Math. 2]

$$\begin{matrix}{SO}_{2} ( r ) = \frac{[ {HbO}_{2} ]}{[ {HbO}_{2} ] + [ Hb ]} \\ = \frac{\frac{{μ}_{a}^{{λ}_{2}} ( r )}{{μ}_{a}^{{λ}_{1}} ( r )} \cdot {ɛ}_{Hb}^{{λ}_{1}} + {ɛ}_{Hb}^{{λ}_{2}}}{( {ɛ}_{HbO}^{{λ}_{2}} - {ɛ}_{Hb}^{{λ}_{2}} ) - \frac{{μ}_{a}^{{λ}_{2}} ( r )}{{μ}_{a}^{{λ}_{1}} ( r )} \cdot ( {ɛ}_{HbO}^{{λ}_{1}} - {ɛ}_{Hb}^{{λ}_{1}} )}\end{matrix} \qquad ( 2 )$$

In the expression, an absorption coefficient at a wavelength λ₁ is denoted by μₐ^{λ}^{₁}, and an absorption coefficient at a wavelength λ₂ is denoted by μₐ^{λ}^{₂}. A molar extinction coefficient for the oxyhemoglobin at the wavelength λ₁ is denoted by ε_{HbO}^{λ}^{₁}, and a molar extinction coefficient for the deoxyhemoglobin at the wavelength λ₁ is denoted by ε_{Hb}^{λ}^{₁}. A molar extinction coefficient for the oxyhemoglobin at the wavelength λ₂ is denoted by ε_{HbO}^{λ}^{₂}, and a molar extinction coefficient for the deoxyhemoglobin at the wavelength λ₂ is denoted by ε_{Hb}^{λ}^{₂}. The ε_{HbO}^{λ}^{₁}, ε_{HbO}^{λ}^{₁}, ε_{HbO}^{λ}^{₂}, and ε_{Hb}^{λ}^{₂} are known values. Position coordinates are denoted by r. The oxygen saturation of blood can be determined based on the ratio of the absorption coefficients at two wavelengths as represented by Math. 2.

Based on Math. 1, the value of the ratio of absorption coefficients at two wavelengths is represented as follows.

[Math. 3]

$$\frac{{μ}_{a}^{{λ}_{2}} ( r )}{{μ}_{a}^{{λ}_{1}} ( r )} = \frac{{P}_{0}^{{λ}_{2}} / {Φ}^{{λ}_{2}}}{{P}_{0}^{{λ}_{1}} / {Φ}^{{λ}_{1}}} \qquad ( 3 )$$

That is, Math. 3 indicates that, in theory, the amount of light Φ^{λ}^{₁} at the wavelength λ₁ and the amount of light Φ^{λ}^{₂} at the wavelength λ2 need to be determined in order to obtain the value of the ratio of the absorption coefficients.

When light at three or more wavelengths is used for measurement, information on the concentration such as the oxygen saturation may be calculated by fitting light absorption distribution data at each measurement wavelength by the method of least squares and the like using the absorbance spectra for the oxyhemoglobin and the deoxyhemoglobin. In this case, the amount of light e at each wavelength also needs to be determined.

Now, a detailed description will be given in conjunction with a case where the apparatus is a photoacoustic microscope. An area measured by the photoacoustic microscope is close to a surface of the object, and thus, the intensity of the PA signal is significantly affected by the beam profile and irradiation position, on the object surface, of excitation light radiated to the object. In a wavelength-variable laser apparatus, the beam profile and irradiation position of excitation light on the object surface may vary according to the wavelength due to differences in the beam profile of light emitted from a laser according to the wavelength and differences in refractive index according to the wavelength. In such a case, the PA signal intensity at each measurement wavelength is affected.

Thus, in order to allow, for example, the value of the oxygen saturation to be accurately determined, the absorption coefficient needs to be determined with the above-described effects at each wavelength taken into account. For example, with two wavelengths, not only the amount of irradiation light is measured but also the beam profile and the irradiation position are measured at each wavelength. Then, the effects of these light irradiation characteristics on the photoacoustic signal are calculated and corrected to allow the ratio of the absorption coefficients to be accurately determined. However, it may be difficult to measure the beam profile and the irradiation position and calculate the effects of the beam profile and the irradiation position on the photoacoustic signal. The position and accuracy of light irradiation vary depending on the presence or absence of the object, the presence or absence of a matching material, and the degree of light reflection associated with presence or absence of the object or the matching material. In such a case, it is difficult to estimate the amount of light Φ at an accuracy otherwise achieved using a light profile. For example, light focusing involves focusing light using an optical lens or the like, making measurement of the beam profile of light radiated to the object difficult. Typically, measurement of the beam profile of light of 10 μm or less in size is difficult. Measurement of the beam profile of light of 1 μm or less in size is more difficult.

### Embodiment 1

A configuration of an object information acquiring apparatus in a first embodiment and a process executed by the object information acquiring apparatus will be described below. The same components are in principle denoted by the same reference numerals, and duplicate description of the components is omitted.

(General Apparatus Configuration)

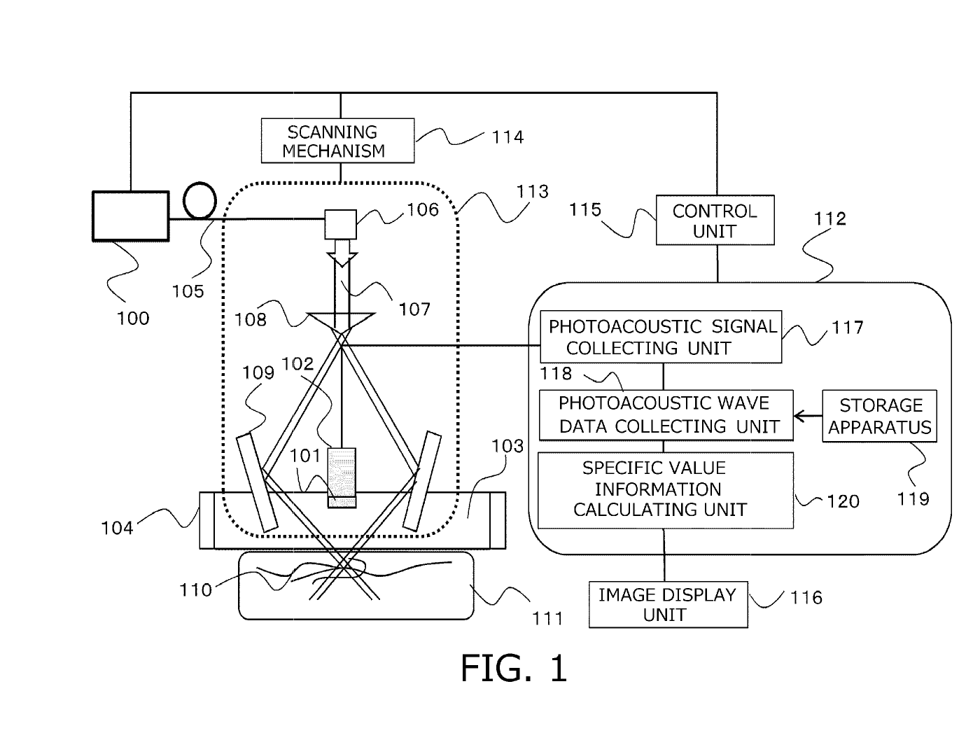

FIG. 1 is a schematic diagram depicting a configuration of a photoacoustic apparatus in the present embodiment. The photoacoustic apparatus in the present embodiment includes a light source 100, a focusing probe 102 including a conversion element 101 that receives photoacoustic waves, and a container 104 filled with an acoustic matching medium 103.

Light from the light source 100 is guided to a light emitting unit 106 by a light waveguide unit 105. The light source 100 outputs a plurality of pulsed light beams with different wavelengths at different timings. At least two wavelengths, a first wavelength and a second wavelength, are needed to determine the oxygen saturation. Light 107 emitted from the light emitting unit 106 is radiated to an object 111 so as to be condensed near a light absorber 110 that is a target segment, by an optical member 108 and an optical member 109.

The light absorber 110 is typically a blood vessel in the living organism and particularly a substance such as hemoglobin which is present in the blood vessel. The light absorber 110 absorbs the energy of light at each of the different wavelengths to generate the corresponding photoacoustic wave. The resultant photoacoustic wave propagates through the object to reach the conversion element 101.

The conversion element 101 outputs reception signals in a time series by focusing and receiving photoacoustic waves. The conversion element 101 preferably includes such an acoustic lens as focuses an acoustic wave. Output reception signals are input to a signal processing unit 112. Reception signals resulting from radiated pulsed light are sequentially input to the signal processing unit 112.

The photoacoustic apparatus also includes a scanning mechanism 114 for making measurement while scanning a measurement unit 113 including the probe 102, the light emitting unit 106, and the like.

The photoacoustic apparatus also includes a control unit 115 configured to control component blocks. The control unit 115 supplies control signals and data needed for the component blocks. Specifically, the control unit 115 supplies a signal that instructs the light source 100 to emit light, reception control signals for the conversion element 101, and control signals for the scanning mechanism 114. Furthermore, the control unit 115, for example, controls signal amplification executed by the signal processing unit 112, AD conversion timings, and storage of reception signals and generates specific-value information on the inside of the object.

The signal processing unit 112 generates specific-value information on the inside of the object 111 using an input reception signal at each wavelength and a transformation coefficient for a conversion between a photoacoustic signal at each wavelength and absorbance which coefficient is determined by measuring a standard sample with a known absorption spectrum. The specific-value information on the inside of the object generated by the signal processing unit 112 is output to an image display unit 116 as image data.

(Internal Configuration of the Signal Processing Unit)

Now, an internal configuration of the signal processing unit 112 in the present embodiment will be described. The signal processing unit 112 in the present embodiment includes a photoacoustic-signal collecting unit 117, a photoacoustic-wave data correcting unit 118, and a specific-value information calculating unit 120.

The photoacoustic-signal collecting unit 117 collects analog reception signals in the time series output from the conversion element 101, and executes signal processing such as amplification of reception signals, AD conversion of analog reception signals, and storage of digitalized reception signals. For example, with two wavelengths, a first electric signal derived from a first wavelength and a second electric signal derived from a second wavelength are obtained.

The photoacoustic-wave data correcting unit 118 acquires light absorption spectrum information on the light absorber 110 in the object 111 for each position thereof using reception signals output from the photoacoustic-signal collecting unit 117 and transformation coefficients in a lookup table stored in a storage apparatus 119. The transformation coefficient is indicative of a relation between a photoacoustic signal at each wavelength and the absorbance. In the embodiments described below, the photoacoustic wave data correcting unit corresponds to a transformation coefficient acquiring unit in the present invention. Therefore, when the light source radiates the first wavelength and the second wavelength, at least a first transformation coefficient and a second transformation coefficient are present which correspond to the respective wavelengths.

The specific-value information calculating unit 120 acquires specific-value information on the inside of the object for each position thereof from a light absorption spectrum distribution information obtained by the photoacoustic-wave data correcting unit 118. The processing executed by the photoacoustic-signal collecting unit and the specific-value information calculating unit are corresponding to the processing executed by a signal processing unit of the present invention.

(Processing in the Signal Processing Unit)

In the present embodiment, the signal processing unit 112 at least obtains information indicative of the oxygen saturation as specific-value information. The “oxygen saturation” is one of the “pieces of information on the concentration” in the specification, and is indicative of the rate of hemoglobin bounded to oxygen in the total hemoglobin in the red blood cells.

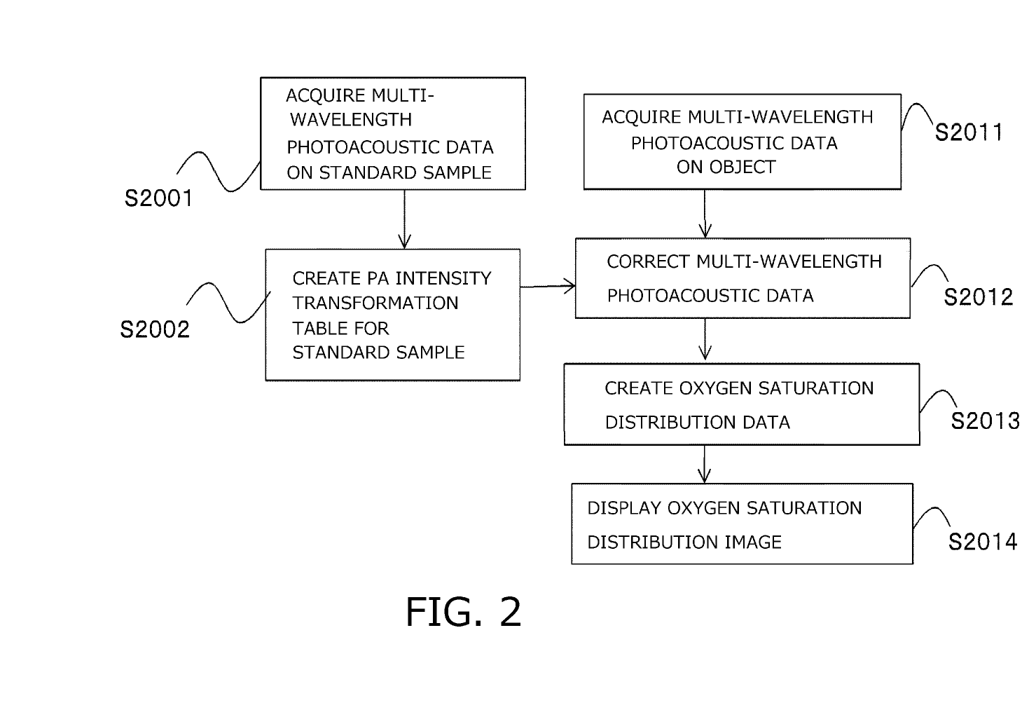

Now, a process flow in which the signal processing unit 112 determines an oxygen saturation distribution will be described using FIG. 2. In step S2011, light at a plurality of wavelengths is radiated to the object at different timings. Consequently, a photoacoustic wave is emitted from the inside of the object and reaches the conversion element 101, which converts the photoacoustic wave into an analog reception signal. The photoacoustic-signal collecting unit 117 collects analog reception signals in the time series output from the conversion element 101 to execute signal processing such as amplification of the reception signals, AD conversion of the analog reception signals, and storage of digitalized reception signals. During measurement, the scanning mechanism 114 moves the probe 102 and a light irradiation spot relative to the object 111 to collect the analog reception signals at a plurality of scanning positions.

In step S2012, the photoacoustic-wave data correcting unit 118 acquires light absorption spectrum distribution information on the light absorber 110 in the object 111 using the reception signals output from the photoacoustic-signal collecting unit 117 and the transformation coefficients.

The transformation coefficient as used herein refers to a transformation coefficient for a conversion between a photoacoustic wave at each wavelength and the absorbance (or the absorption coefficient). The transformation coefficients are stored in the storage apparatus 119 as a lookup table. The lookup table has the transformation coefficients for the wavelengths used to measure the object 111. That is, the lookup table saved in the storage apparatus is a lookup table indicative of relations between the light wavelengths and the transformation coefficients.

When the object 111 is measured, the lookup table is preferably pre-created. However, the lookup table may be created after the object 111 is measured. The transformation coefficient is calculated by measuring a standard sample with a known absorption spectrum at each of the wavelengths used to measure the object 111 (steps S2001 to S2002).

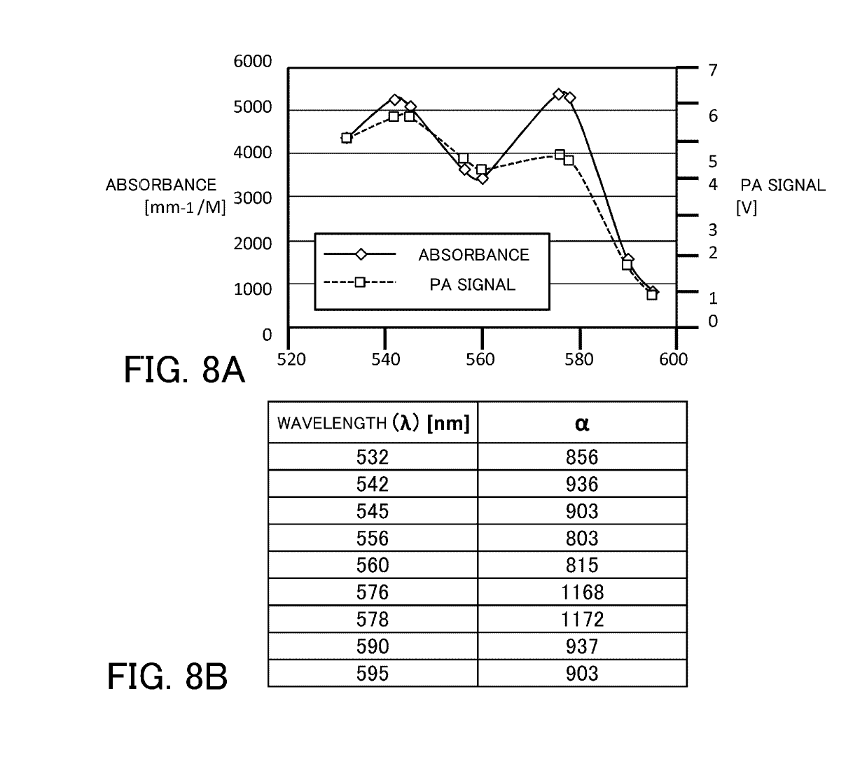

A method in which, before photoacoustic measurement, the lookup table is created by determining the transformation coefficients will be described below with reference to FIG. 8 as needed. A transformation coefficient α^{λ}^{ᵢ} is represented by the following expression. The superscript λi is indicative of each measurement wavelength.

[Math. 4]

$${α}^{{λ}_{i}} = \frac{{C}^{{λ}_{i}}}{{PA}^{{λ}_{i}}} \qquad ( 4 )$$

In the expression, a coefficient that represents absorption of light at each wavelength by the standard sample is denoted by C^{λ}^{ᵢ}. The coefficient is, for example, the absorbance or the absorption coefficient. A measured photoacoustic signal is denoted by PA^{λ}^{ᵢ}, and as the measured photoacoustic signal, the signal intensity of a reception signal may be used which signal is output from the photoacoustic-signal collecting unit 117 when the standard sample is measured at each wavelength. Examples of expression of the signal intensity of the reception signal include a maximum value for the photoacoustic signal (PEAK value), a difference between the maximum value and a minimum value for the photoacoustic signal (PEAK TO PEAK value), and the maximum value and an integration value for the photoacoustic signal resulting from envelope detection.

When the measurement wavelengths for the object 111 are not predetermined, the lookup table preferably has transformation coefficients for various wavelengths that may be used to measure the object 111. In that case, measurement is made at various wavelengths in wavelength regions near the wavelengths at which measurement may be made, and the transformation coefficient is determined for each of the wavelengths.

FIG. 8A is a graph in which the absorbance and the PA signal intensity are plotted, the absorbance and the PA signal intensity having been obtained when a standard sample with a known configuration was irradiated with light at a plurality of different wavelengths. In this case, the standard sample is blood with a known oxygen saturation of 90%. In FIG. 8A, a left axis of ordinate represents the absorbance, a right axis of ordinate represents the PA signal intensity, and an axis of abscissas represents the wavelength of light. Based on these values, a calculation in Math. 4 is executed to determine a transformation coefficient α for each wavelength.

FIG. 8B is an example of the lookup table stored in the storage apparatus, which serves as a memory. The illustrated table is referenced for the transformation coefficient α for each wavelength when the only parameter of interest is the wavelength. Based on the graph in FIG. 8A and known optical characteristics, transformation coefficients for wavelengths not plotted may be estimated. Instead of or in addition to the lookup table, transformation equations for the absorbance and the PA signal intensity may be determined.

The standard sample used has a known absorption spectrum. The standard sample also preferably simulates the light absorber 110. For example, when the light absorber 110 is a blood vessel, the sample is preferably a fine tube containing a light absorbing material with a known absorption spectrum. Moreover, the light absorbing material preferably flows through the fine tube so as to be prevented from cohering.

The light absorbing material preferably has an absorption coefficient close to the absorption coefficient of the light absorber 110. The light absorbing material further preferably allows light scattering similar to light scattering allowed by the light absorber 110. Such a light absorbing material is, for example, blood. Acquisition of the absorption spectrum of the blood used involves the need to pre-measure the oxygen saturation of the blood used and to calculate the absorption spectrum from the absorbance of oxyhemoglobin and deoxyhemoglobin. Alternatively, the absorption spectrum may be calculated based on measurements made using a spectrometer or the like.

Other examples of the light absorbing material may include diluted India ink and a dye solution. Furthermore, intralipid or titanium oxide may be used to additionally allow light scattering.

The standard sample need not be a liquid. For example, a sample may be used which is obtained by gelling a gel mixed with India ink, a dye, or the like. An example of such a gel is a urethane gel. Agar or gelatin may also used as the standard sample.

The photoacoustic-wave data correcting unit 118 reads the transformation coefficient for each measurement wavelength from the lookup table in the storage apparatus 119. Then, as represented by the following expression, the photoacoustic-wave data correcting unit 118 calculates the product of the transformation coefficient and a reception signal PA_{OBJ}^{λ}^{ᵢ} output from the photoacoustic-signal collecting unit 117 while the object 111 is measured to obtain light absorption information C_{OBJ}^{λ}^{ᵢ}.

[Math. 5]

C_{OBJ}^{λ}^{ᵢ}=α^{λ}^{ᵢ}·PA_{OBJ}^{λ}^{ᵢ} (5)

Such a calculation allows the beam profile and the amount of light at each wavelength to be corrected. As a result, the light absorption spectrum information on the light absorber 110 in the object 111 can be acquired. Light absorption spectrum distribution information on the light absorber 110 in the object 111 is obtained by executing the above-described calculation for each measurement position.

In the present embodiment, the transformation coefficient for each measurement wavelength is acquired by referencing the lookup table stored in the storage apparatus. However, transformation equations α(λ) using the wavelength as a variable may be stored in the photoacoustic-wave data correcting unit 118 so as to allow the value to be determined by a transformation calculation. The transformation equation α(λ) is a relational expression that represents the relation between the wavelength of light and the transformation coefficient. That is, the photoacoustic-wave data correcting unit 118 may acquire the transformation coefficient corresponding to the wavelength of light used in accordance with the transformation equation α(λ), which represents the relation between the wavelength of light and the transformation coefficient.

To allow the transformation equation α(λ) to be calculated, a standard sample with a known absorption spectrum is measured at variable wavelengths near the wavelengths that may be used for measurement, using the photoacoustic apparatus in the present embodiment. Then, measurement results are applied to Math. 4 to determine transformation coefficients. Then, based on each of the resultant transformation coefficients for the various wavelengths, the transformation equation α(λ) using the wavelength as a variable is derived using the method of least squares and the like. The standard sample used is similar to the standard sample used to create a lookup table.

In step S2013, the specific-value information calculating unit 120 acquires oxygen saturation distribution information on the light absorber 110 using the light absorption spectrum distribution information on the light absorber 110 obtained by the photoacoustic-wave data correcting unit 118.

For example, a case of two measurement wavelengths will be described. In this case, the photoacoustic-wave data correcting unit 118 acquires two pieces of light absorption spectrum information on the absorber 110 for each position thereof, C_{OBJ}^{λ}^{₁}(r) and C_{OBJ}^{λ}^{₂}(r). Spatial coordinates are denoted by r; data resulting from conversion of a time axis direction in the light absorption information C_{OBJ}^{λ}^{ᵢ} into a depth direction is plotted on the spatial coordinates. Then, the oxygen saturation for each position is calculated using Math. 6 in which μₐ^{λ}^{₁}(r) and μₐ^{λ}^{₂}(r) are replaced with C_{OBJ}^{λ}^{₁}(r) and C_{OBJ}^{λ}^{₂} (r).

[Math. 6]

$$\begin{matrix}{( S O )}_{2} ( r ) = \frac{[ {HbO}_{2} ]}{[ {HbO}_{2} ] + [ Hb ]} \\ = \frac{\frac{{C}_{OBJ}^{{λ}_{2}} ( r )}{{C}_{OBJ}^{{λ}_{1}} ( r )} \cdot {ɛ}_{Hb}^{{λ}_{1}} - {ɛ}_{Hb}^{{λ}_{2}}}{( {ɛ}_{HbO}^{{λ}_{2}} - {ɛ}_{Hb}^{{λ}_{2}} ) - \frac{{C}_{OBJ}^{{λ}_{2}} ( r )}{{C}_{OBJ}^{{λ}_{1}} ( r )} \cdot ( {ɛ}_{HbO}^{{λ}_{1}} - {ɛ}_{Hb}^{{λ}_{1}} )}\end{matrix} \qquad ( 6 )$$

With three or more measurement wavelengths, the oxygen saturation is calculated by comparing the light absorption spectrum information on the absorber 110 obtained by the photoacoustic-wave data correcting unit 118 with the light absorption spectra of the oxyhemoglobin and the deoxyhemoglobin. For example, the oxygen saturation is calculated by using the concentrations of the oxyhemoglobin and the deoxyhemoglobin as parameters and using, for example, fitting based on the method of least squares. If the light absorber 110 contains absorbers other than the oxyhemoglobin and the deoxyhemoglobin and the effects of light absorption by these absorbers are measurably significant, the fitting preferably involves the light absorption spectra of the absorbers. In this case, parameters for the fitting are the concentration of the oxyhemoglobin and the deoxyhemoglobin and the concentration of the absorbers. The absorbers may be, for example, melanin, fat, water, or collagen.

The specific-value information calculating unit 120 creates sound pressure distribution data for at least one of the measurement wavelengths using the reception signals obtained in S2011. A technique for creation is, for example, as follows. First, envelope detection is performed on the resultant reception signals with respect to temporal changes, and then, the time axis direction in signals for the respective light pulses is converted into the depth direction, and the resultant data is plotted on the spatial coordinates. This is performed for each scanning position to provide sound pressure distribution data. Such signal processing is common in photoacoustic microscopes. This processing method allows reception signals to be directly plotted, enabling a reduction in arithmetic loads and time needed. However, a reconstruction technique may be used to acquire sound pressure.

In step S2014, an oxygen saturation distribution image is displayed on the image display unit 116 using the oxygen saturation distribution data and the sound pressure distribution data created by the specific-value information calculating unit 120. As a display technique, a method is available which involves determining, for each voxel, chromaticity and lightness based on the oxygen saturation distribution data and the sound pressure distribution data, respectively, and displaying an image based on the chromaticity and the lightness. A technique is also available which involves displaying the oxygen saturation distribution data only for voxels with a sound pressure intensity equal to or higher than a predetermined threshold.

(Component Examples in the Signal Processing Unit)

Component examples in the signal processing unit 112 in the present embodiment will be described below in detail.

As the photoacoustic-signal collecting unit 117, a circuit may be used which is generally referred to as a DAS (Data Acquisition System). Specifically, the photoacoustic-signal collecting unit 117 includes an amplifier that amplifies reception signals, an AD converter that digitalizes analog reception signals, and, for example, a memory such as a FIFO or a RAM which stores reception signals.

As each of the photoacoustic-wave data correcting unit 118 and the specific-value information calculating unit 120, a processor such as a CPU or a GPU or an arithmetic circuit such as an FPGA (Field Programmable Gate Array) chip may be utilized. The photoacoustic-wave data correcting unit 118 and the specific-value information calculating unit 120 may each include a plurality of processors or arithmetic circuits instead of a single processor or arithmetic circuit.

The photoacoustic-signal collecting unit 117 may include a memory that stores reception signals. The memory typically includes any of storage media such as a ROM, a RAM, and a hard disk. The memory may include a plurality of storage media instead of a single storage medium.

The photoacoustic-wave data correcting unit 118 and the specific-value information calculating unit 120 can reference the storage apparatus 119, which stores the lookup table for transformation coefficients, reception signals, generated distribution data and display image data, various measurement parameters, and the like. The storage apparatus typically includes one or more storage media such as a ROM, a RAM, and a hard disk.

Now, specific examples of components other than the components in the signal processing unit 112 will be described.

### (Light Source 100)

The light source 100 is preferably a pulsed light source that can generate pulsed light with a pulse width of the order of nanoseconds to microseconds. A specific pulse width is approximately 1 to 100 nanoseconds. The wavelength of the pulsed light ranges from approximately 200 nm to approximately 1600 nm. In particular, a visible light region is preferably used when the blood vessel near the surface of the living organism is imaged at a high resolution as in the present embodiment. However, a terahertz wave region, a microwave region, or a radio wave region may also be used.

The specific light absorber 110 is preferably a laser. In particular, when implementing measurement in use of light at a plurality of wavelengths, a laser that enables conversion of an oscillation wavelength is preferable. However, any laser may be used so long as the object 111 can be irradiated with light at a plurality of wavelengths, and thus, a plurality of lasers oscillating light at different wavelengths may be used while each switching the oscillation or alternately radiating the light. Even when a plurality of lasers is used, the lasers are collectively represented as the light source.

As the laser, any of various lasers such as a solid laser, a gas laser, a dye laser, and a semiconductor laser may be used. In particular, a pulse laser such as an Nd:YAG laser or an alexandrite laser is preferable. A Ti:sa laser or an OPO (Optical Parametric Oscillators) laser may be used which uses Nd:YAG laser light as excitation light. Instead of the laser, a flash lamp or a light-emitting diode and the like may be used.

(Optical System)

Light is transmitted from the light source 100 to the object 111 by the light waveguide unit 105 and the light emitting unit 106. As each of the light waveguide unit 105 and the light emitting unit 106, an optical element such as an optical lens, a mirror, or an optical fiber may be used. However, the light source 100 may directly irradiate the object with light. The optical member 108 may be an axicon mirror, and the optical member 109 may be an optical mirror or the like.

For example, as the optical system, an optical lens may be adopted which focuses light radiated to the object 111 as a light spot of 10 μm or less in size. Alternatively, as the optical system, an optical lens may be adopted which focuses light radiated to the object 111 so as to reduce the light spot to 1 μm or less in size.

(Probe 102)

The probe 102 includes one or more conversion elements 101. Examples of the conversion element 101 include a piezoelectric element such as lead zirconate titanate (PZT) which uses a piezoelectric phenomenon, a conversion element using light resonance, or a capacitive conversion element such as a CMUT. Any other conversion element may be used so long as the conversion element can receive and convert acoustic waves into electric signals. When the probe 102 includes a plurality of conversion elements 101, the conversion elements 115 are preferably arranged in a plane or a curved plane, and in this case, referred to as a 1 D array, a 1.5 D array, a 1.75 D array, or a 2 D array.

In the case of a photoacoustic microscope, the probe 102 is preferably a focusing probe. For example, the conversion element 101 is provided with an acoustic lens to allow acoustic waves to be converged. In the probe 102, an amplifier may be provided which amplifies analog signals output from the conversion element 101.

A reception unit of the probe 102 needs to be in contact with the acoustic matching medium 103 in the container 104. As the acoustic matching medium, water, matching gel, or the like may be used. To allow photoacoustic waves to be transmitted through the container 104, a bottom surface of container 104 is preferably a film. The film used preferably allows only insignificant light absorption and scattering.

(Display Unit 116)

As the display unit 116, a display such as an LCD (Liquid Crystal Display), a CRT (Cathode Ray Tube), or an organic EL display may be utilized. Instead of being installed in the photoacoustic apparatus in the present embodiment, the display unit 116 may be prepared separately from the photoacoustic apparatus and connected to the photoacoustic apparatus.

[Variation]

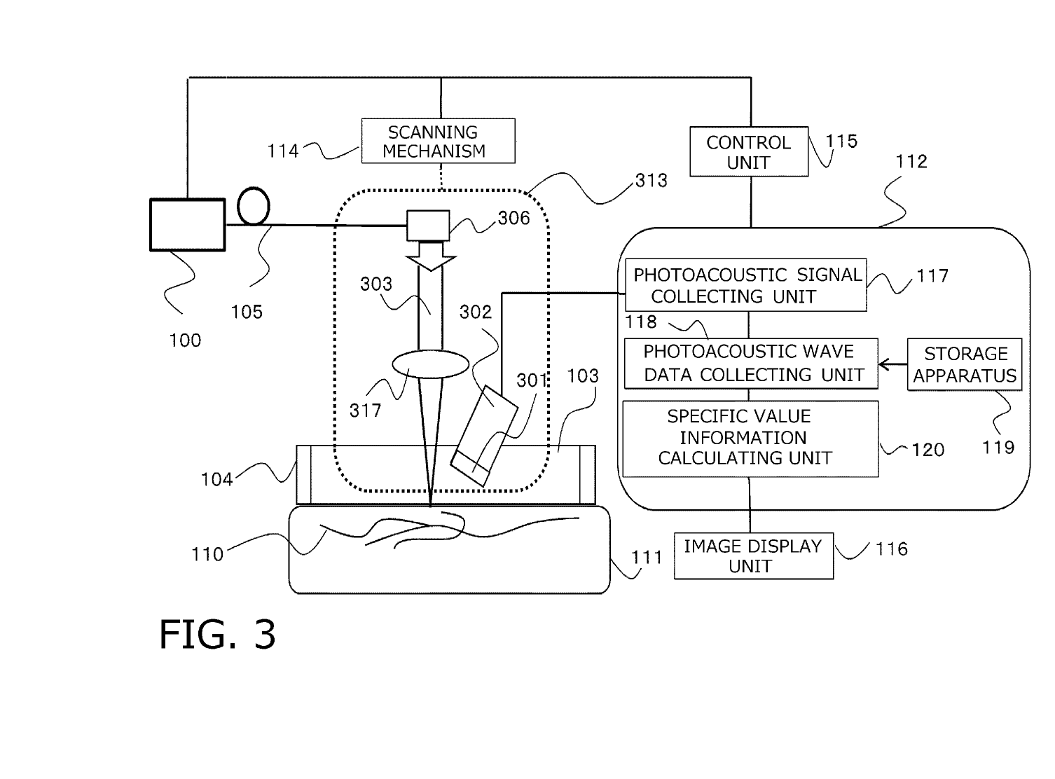

In the above description, the photoacoustic microscope achieves a high resolution by using the focusing probe 102 to focus acoustic waves. However, the present embodiment can be used for photoacoustic apparatuses other than the photoacoustic microscope. FIG. 3 depicts a configuration example in which the present invention is used for a photoacoustic microscope that irradiates the object with light focused on the object to acquire a photoacoustic image with a high resolution. Components of the probe 102 other than those included in a measurement unit 313 are similar to the corresponding components in FIG. 1. These components are denoted by the same reference numerals and will not be described below in detail.

Light 303 emitted from the light emitting unit 306 is condensed at a target segment in the object 111 by an optical lens 317. The optical lens 317 is preferably an objective lens. When the light absorber 110 absorbs the energy of light, a photoacoustic wave is generated at a condensed-light spot. The resultant photoacoustic wave propagates through the object and reaches the conversion element 301. A subsequent process flow can be executed as in the case of the above-described acoustic focusing probe.

The use of this configuration for measurement allows the specific-value information on the inside of the object 111 to be acquired at a high resolution. In this case, the effects of the beam profile and the amount of light at each wavelength of light radiated to the object can also be corrected. As a result, also in the present variation, the effect of the present invention can be enjoyed in which the specific-value information such as the oxygen saturation is easily and accurately obtained.

### Embodiment 2

A configuration of an object information acquiring apparatus in Embodiment 2 of the present invention and a process executed by the object information acquiring apparatus will be described.

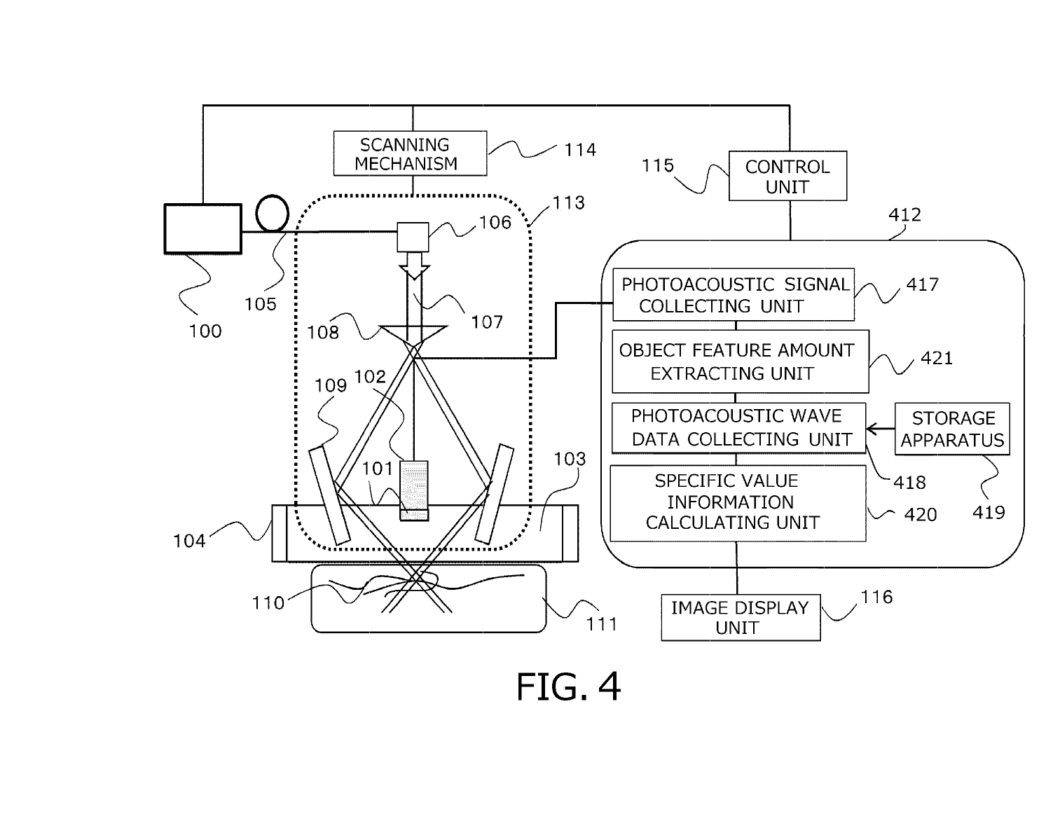

FIG. 4 is a schematic diagram depicting a configuration of a photoacoustic apparatus in the present embodiment. Components of the photoacoustic apparatus other than those included in a signal processing unit 412 are common to FIG. 1. These components are denoted by the same reference numerals and will not be described in detail.

(Internal Configuration of the Signal Processing Unit)

Components in the signal processing unit 412 in the present embodiment will be described. The signal processing unit 412 in the present embodiment includes a photoacoustic-signal collecting unit 417, a photoacoustic-wave data correcting unit 418, a specific-value information calculating unit 420, and an object feature amount extracting unit 421.

The photoacoustic-signal collecting unit 417 collects analog reception signals in the time series output from the conversion element 101, and executes signal processing such as amplification of reception signals, AD conversion of analog reception signals, and storage of digitalized reception signals.

The object feature amount extracting unit 421 creates sound pressure distribution data using reception signals output from the photoacoustic-signal collecting unit 417, and extracts object feature amounts for each position from the resultant sound pressure distribution data. The object feature amount extracting unit corresponds to a feature amount acquiring unit in the present invention.

The photoacoustic-wave data correcting unit 418 acquires the light absorption spectrum information on the light absorber 110 in the object 111 for each position thereof using the reception signals output from the photoacoustic-signal collecting unit 417, the object feature amounts extracted by the object feature amount extracting unit 421, and the transformation coefficients. The transformation coefficients are transformation equations for the photoacoustic signal and the absorbance at each wavelength which are present as the lookup table stored in the storage apparatus 419. The specific-value information calculating unit 420 acquires the specific-value information on the inside of the object for each position from the light absorption spectrum distribution information obtained by the photoacoustic-wave data correcting unit 418.

(Processing in the Signal Processing Unit)

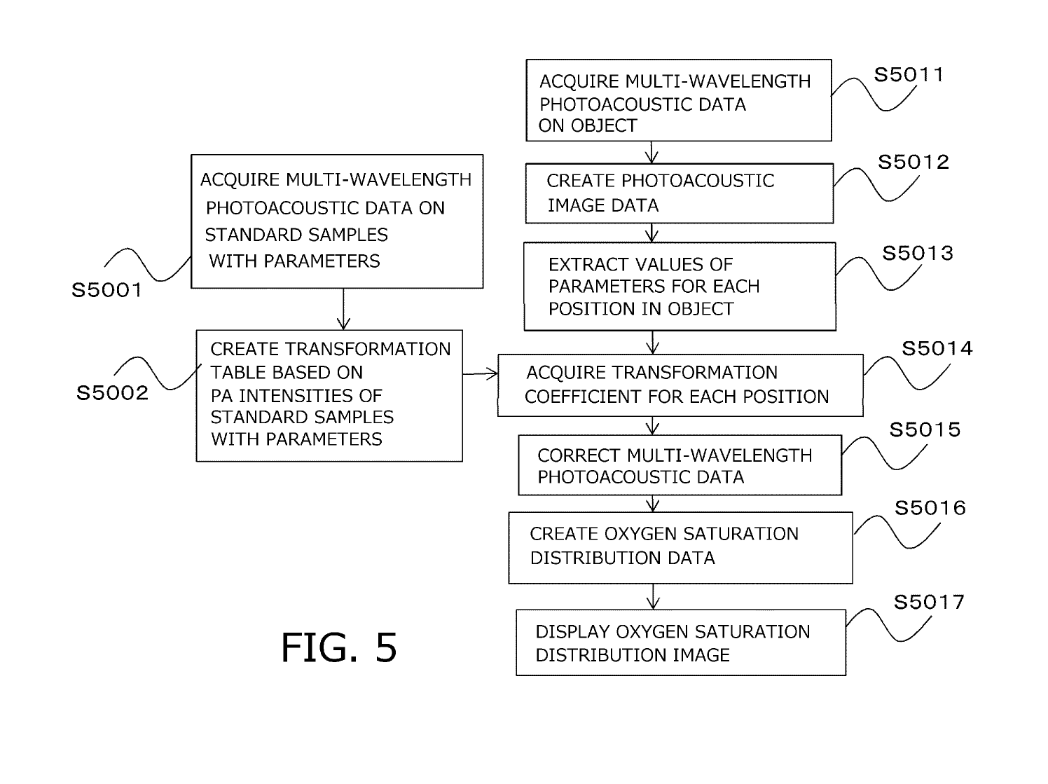

Now, a process flow in which the signal processing unit 412 determines the oxygen saturation distribution will be described using FIG. 5. In step S5011, light at a plurality of wavelengths is radiated at different timings, and then, the photoacoustic-signal collecting unit 417 collects analog reception signals in the time series output from the conversion element 101 for each measurement wavelength. The photoacoustic-signal collecting unit 417 then executes signal processing such as amplification of reception signals, AD conversion of analog reception signals, and storage of digitalized reception signals. During measurement, the scanning mechanism 114 moves the probe 102 and the light irradiation spot relative to the object 111 to collect analog reception signals at a plurality of scanning positions.

In step S5012, the object feature amount extracting unit 421 creates photoacoustic image data (hereinafter referred to as sound pressure distribution data) using the reception signals output from the photoacoustic-signal collecting unit 417. The sound pressure distribution data is created for at least one of the measurement wavelengths. An example of a method for creating sound pressure distribution data will be described. First, envelope detection is performed on the resultant reception signals with respect to temporal changes, and then, the time axis direction in signals for the respective light pulses is converted into the depth direction, and the resultant data is plotted on the spatial coordinates. This is performed for each scanning position to provide sound pressure distribution data.

In step S5013, the object feature amount extracting unit 421 extracts object feature amounts from the resultant sound pressure distribution data. The object feature amounts refer to the values of parameters for the light absorber 110 obtained at each position. Examples of the parameters include the thickness, angle (the angle to a normal direction of a scanning direction), and shape of the light absorber 110 at each position in the object 111, and a depth from the object surface. The parameters are extracted as follows: at a certain position in the object 111, a blood vessel of 100 μm in thickness is present at a depth of 3 mm from the surface so as to incline at an angle of 40 degrees. A known information processing technique such as a pattern matching process may be utilized to extract the thickness and the angle.

In the example described in the present embodiment, the object feature amount extracting unit 421 extracts the object feature amounts from the photoacoustic image data. However, the object feature amount extracting unit 421 may use any method to acquire the object feature amounts so long as the object feature amount extracting unit 421 can acquire the object feature amounts. For example, the object feature amount extracting unit 421 may acquire object feature amounts from image data obtained by modality (for example, an ultrasonic diagnosis apparatus, an MRI, or a CT) other than the photoacoustic apparatuses.

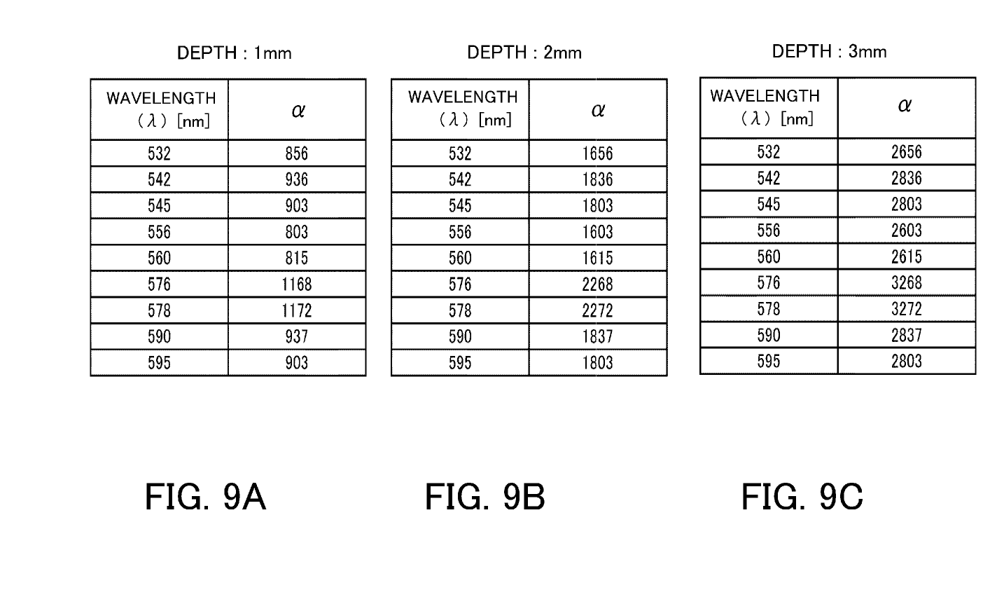

The effects of the various parameters on the photoacoustic signal will be discussed. Tables in FIG. 9A, FIG. 9B, and FIG. 9C indicate the effects of the depth of the blood vessel on the photoacoustic signal. An increased depth of position of the blood vessel increases the amount of light attenuation in a background tissue (fat or the like) before the light reaches the blood vessel, while reducing the magnitude of the photoacoustic signal. Therefore, even when the same light absorber is a measurement target, the value of a increases consistently with the depth from the object surface. The light absorption coefficient for the background tissue varies according to the wavelength, and thus, a difference in light attenuation amount at each wavelength increases consistently with the depth. Hence, preferably, tables are created for the respective depths and stored in the storage apparatus.

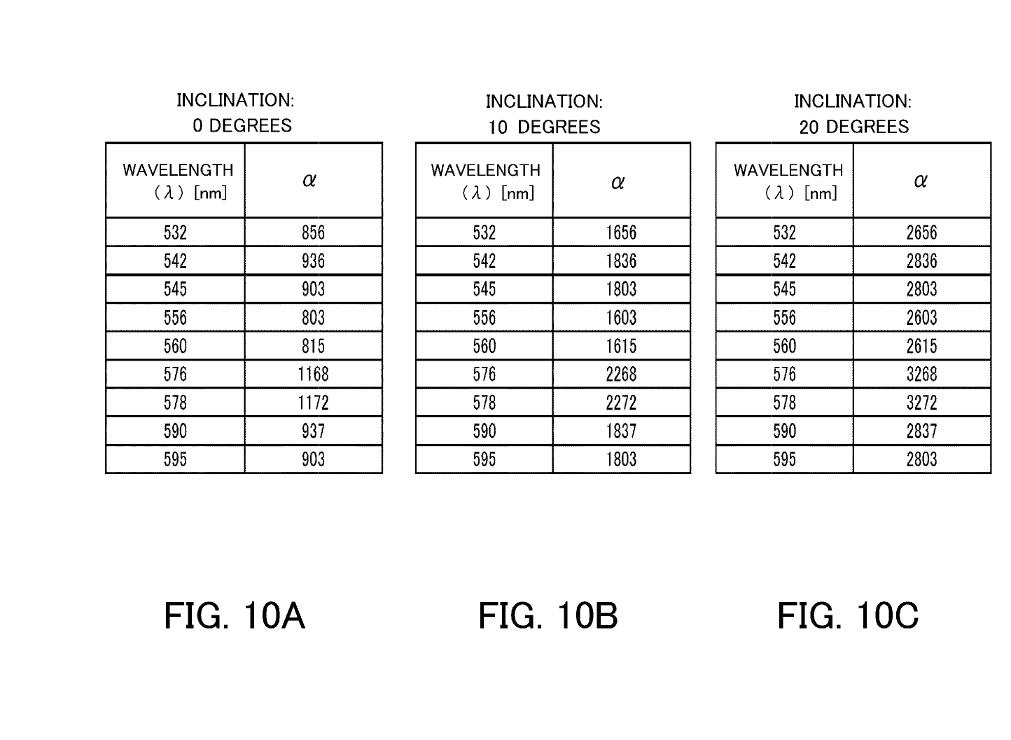

Tables in FIG. 10A, FIG. 10B, and FIG. 10C indicate the effects of the inclination of the blood vessel to the orientation of the probe on the photoacoustic signal. The orientation of the probe may be considered to be, for example, a direction in which the probe exhibits a high reception sensitivity. For a two-dimensional array probe, the orientation of the probe may be considered to be a normal direction of a reception surface. Normally, the intensity of the reception signal decreases with increasing inclination of the incident direction of the photoacoustic wave from the direction in which the probe exhibits a high reception sensitivity. Therefore, the value of a increases consistently with the inclination angle.

For the thickness, the signal intensity normally increases consistently with thickness of the blood vessel. However, a change in thickness also changes the frequency characteristics of the resultant photoacoustic wave. The intensity of the reception signal is also affected by a relation between the photoacoustic wave and a frequency band of the probe. Therefore, when a table is created taking the thickness of the blood vessel into account as a parameter, blood vessel phantoms with various thicknesses are preferably measured at each wavelength, with transformation coefficients determined based on the results of the measurement.

The number of dimensions of a table increases consistently with the number of those of the above-described various parameters which are used to create the table. For example, when the depth and the inclination are used, a three-dimensional table for the depth and the inclination plus the wavelength is created and held. This involves the need to perform preliminary information acquisition with the three dimensions varied to determine transformation coefficients. Hence, parameter items may be set taking into account time and effort needed to perform the information acquisition before actual photoacoustic measurement and the magnitude of effects on the signal intensity. For the range of parameter values, the range of realistic values may be set.

In S5014, the photoacoustic-wave data correcting unit 418 acquires the transformation coefficient for the photoacoustic signal and the absorbance (or the absorption coefficient) at each wavelength for each position, based on the lookup table in the storage apparatus 419 and the value of the parameter for the light absorber 110 at the position extracted in S5013. The lookup table has the transformation coefficients for the wavelengths used to measure the object 111. Each of the transformation coefficients has a value that uses, as a variable, at least one of the above-described parameters, that is, the thickness, angle, and depth of the light absorber 110. That is, the lookup table in the present embodiment represents the relations between the wavelength of light and the feature amounts such as the thickness, angle, and depth of the light absorber 110 and the transformation coefficients.

When the object 111 is measured, the lookup table is preferably pre-created. However, the lookup table may be created after the object 111 is measured.

The transformation coefficients are calculated by measuring a standard sample with a known absorption spectrum using the photoacoustic apparatus in the present embodiment. The standard sample is measured at the wavelengths used to measure the object 111. A transformation coefficient α^{λ}^{ᵢ} [w,a,d] is calculated by the expression depicted below.

Each measurement wavelength is denoted by a superscript λᵢ. The thickness, the angle, and the depth are denoted by (w), (a), and (d), respectively. Not all these parameters need to be taken into account but one or two of the parameters may be exclusively taken into account. Furthermore, any other parameter, for example, a shape, may be used.

[Math. 7]

$${α}^{{λ}_{i}} [ w , a , d ] = \frac{{C}^{{λ}_{i}}}{{PA}^{{λ}_{i}} [ w , a , d ]} \qquad ( 7 )$$

A coefficient that represents the absorption of light at each wavelength by the standard sample is denoted by C^{λ}^{ᵢ}. For example, the absorbance or the absorption coefficient may be utilized as the coefficient. A measured photoacoustic signal is denoted by PA^{λ}^{ᵢ}, and as the measured photoacoustic signal, a reception signal may be used which is output from the photoacoustic-signal collecting unit 417 when the standard sample is measured at each wavelength. Examples of expression of the intensity of the reception signal include the maximum value for the photoacoustic signal (PEAK value), the difference between the maximum value and the minimum value for the photoacoustic signal (PEAK TO PEAK value), and the maximum value or the integration value for the photoacoustic signal resulting from envelope detection.

For the standard sample, samples with various thicknesses, angles, and shapes and the like are prepared, and each of the samples is measured at each wavelength. The samples are further measured with the depth thereof varied. Measuring various samples as described above allows acquisition of transformation coefficients corresponding to various values of each parameter. The material for the standard sample and the like are similar to those in Embodiment 1.

The resultant transformation coefficients are stored in the photoacoustic-wave data correcting unit or the memory in the form of a lookup table. Then, the transformation coefficient for the photoacoustic signal and the absorbance (or the absorption coefficient) at each wavelength is acquired for each position by referencing the table based on the value of each parameter for the light absorber 110 for the position extracted in S5013.

In step S5015, the photoacoustic-wave data correcting unit 418 acquires the light absorption spectrum distribution information on the light absorber 110 in the object 111 using the reception signal output from the photoacoustic-signal collecting unit 417 and the transformation coefficients for the respective wavelengths at each position extracted in S5014.

At this time, the light absorption information C_{OBJ}^{λ}^{ᵢ} is obtained by taking the product of the transformation coefficient and the reception signal PA_{OBJ}^{λ}^{ᵢ} output from the photoacoustic-signal collecting unit 117 while the object 111 is measured, as represented by Math. 8.

[Math. 8]

C_{OBJ}^{λ}^{ᵢ}(r)=α^{λ}^{ᵢ}(r)·PA_{OBJ}^{λ}^{ᵢ}(r) (8)

A coordinate position r corresponds to data plotted on the spatial coordinates and resulting from conversion of the time axis direction in the reception signal PA_{OBJ}^{λ}^{ᵢ} for each light pulse into the depth direction.

As a result, the light absorption spectrum information on the light absorber 110 in the object 111 can be acquired in which, for example, the beam profile and the amount of light at each wavelength are simultaneously corrected. This calculation is executed for each measurement position to allow acquisition of the light absorption spectrum information on the light absorber 110 in the object 111.

In step S5016, the specific-value information calculating unit 420 acquires the oxygen saturation distribution information on the light absorber 110 using the light absorption spectrum distribution information on the light absorber 110 obtained in S5015. A processing method for acquiring the oxygen saturation distribution information is similar to the processing method executed in S2013 and will thus not be described.

In step S5017, an oxygen saturation distribution image is displayed on the image display unit 116 using the oxygen saturation distribution data created in S5016 and the sound pressure distribution data created in S5012. A technique for displaying in the oxygen saturation distribution image is similar to the technique used in S2014 and will thus not be described.

(Specific Component Examples in the Signal Processing Unit 112)

Details of component examples in the signal processing unit 412 in the present embodiment are similar to the details of component examples in the signal processing unit 112 in Embodiment 1 except for the object feature amount extracting unit 421. The object feature amount extracting unit 421 may be a processor such as a CPU or a GPU (Graphic Processing Unit), or an arithmetic circuit such as an FPGA (Field Programmable Gate Array) chip. Instead of including a single processor or arithmetic circuit, the object feature amount extracting unit 421 may include a plurality of processors or arithmetic circuits. The object feature amount extracting unit 421 also includes a memory that stores sound pressure distribution data generated, object feature amounts, and the like. The memory typically includes one or more of storage media such as a ROM, a RAM, and a hard disk. The object feature amount extracting unit 421 may have a physical configuration separate from the physical configuration of the signal processing unit 412. The object feature amount extracting unit 421 may be implemented as software running in an information processing apparatus in which the signal processing unit 412 is implemented.

The present embodiment is applicable to apparatus configurations other than the apparatus configuration in FIG. 4. For example, the present embodiment is applicable to any light- or sound-focus photoacoustic microscopes and to any photoacoustic apparatuses that perform image reconstruction.

The use of the photoacoustic apparatus disclosed in the present embodiment allows correction of the effects of the beam profile and the amount of light at each wavelength radiated to the object, enabling specific-value information such as the oxygen saturation to be easily and accurately obtained.

### Embodiment 3

A configuration of an object information acquiring apparatus in Embodiment 3 of the present invention and a process executed by the object information acquiring apparatus will be described.

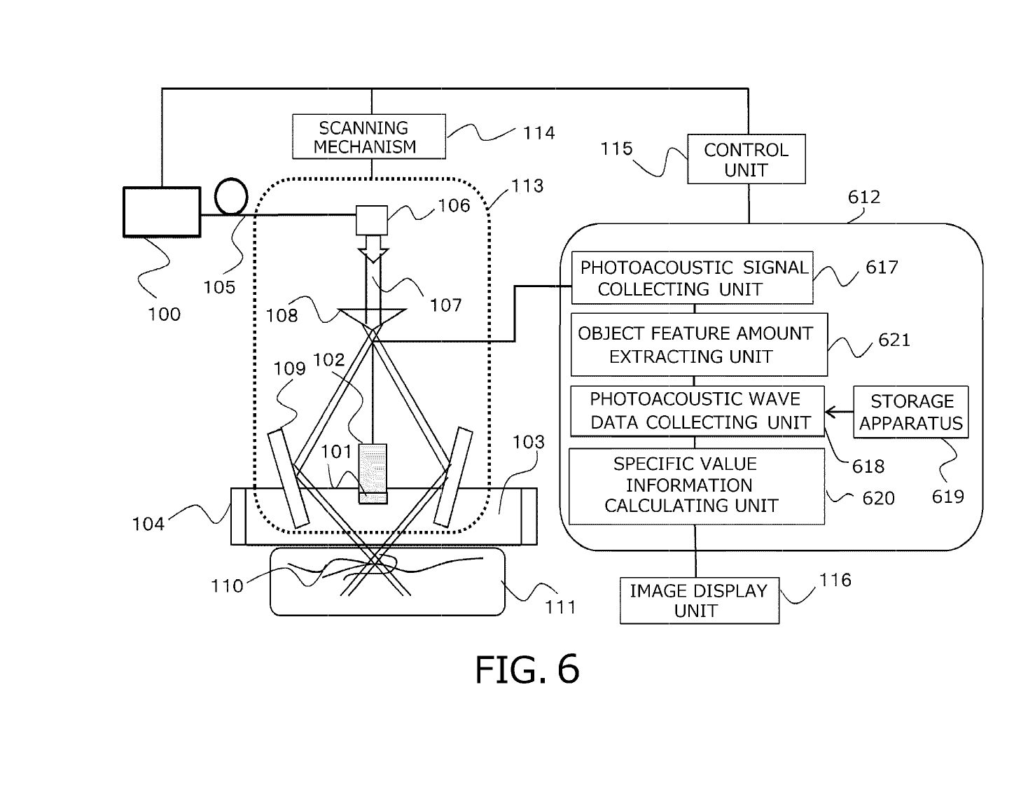

FIG. 6 is a schematic diagram depicting a configuration of a photoacoustic apparatus in the present embodiment. Components of the photoacoustic apparatus are common to the apparatus depicted in FIG. 1 except for the signal processing unit. Thus, the components are denoted by the same reference numerals and will not be described below in detail. For specific examples of the components in the present embodiment and processes executed by the components, those of the specific component examples and the processes which are similar to the corresponding specific component examples and processes in Embodiment 1 and Embodiment 2 will not be described below in detail.

(Internal Configuration of the Signal Processing Unit)

An internal configuration of a signal processing unit 612 in the present embodiment will be described. The signal processing unit 612 in the present embodiment includes a photoacoustic-signal collecting unit 617, a photoacoustic-wave data correcting unit 618, a specific-value information calculating unit 620, and an object feature amount extracting unit 621.

The photoacoustic-signal collecting unit 617 collects analog reception signals in the time series output from the conversion element 101. The photoacoustic-signal collecting unit 617 executes signal processing such as amplification of reception signals, AD conversion of analog reception signals, and storage of digitalized reception signals.

The object feature amount extracting unit 621 creates sound pressure distribution data using reception signals output from the photoacoustic-signal collecting unit 617, and extracts object feature amounts for each position from the resultant sound pressure distribution data.

The photoacoustic-wave data correcting unit 618 acquires the light absorption spectrum of the light absorber 110 in the object 111 for each position thereof using the reception signals from the photoacoustic-signal collecting unit, the extracted object feature amounts, and the transformation coefficients for the photoacoustic signal and the absorbance at the respective wavelengths derived from the corresponding transformation equations in a storage apparatus 619.

The specific-value information calculating unit 620 acquires specific-value information on the inside of the object for each position thereof from the light absorption spectrum distribution information obtained by the photoacoustic-wave data correcting unit 618.

(Processing in the Signal Processing Unit)

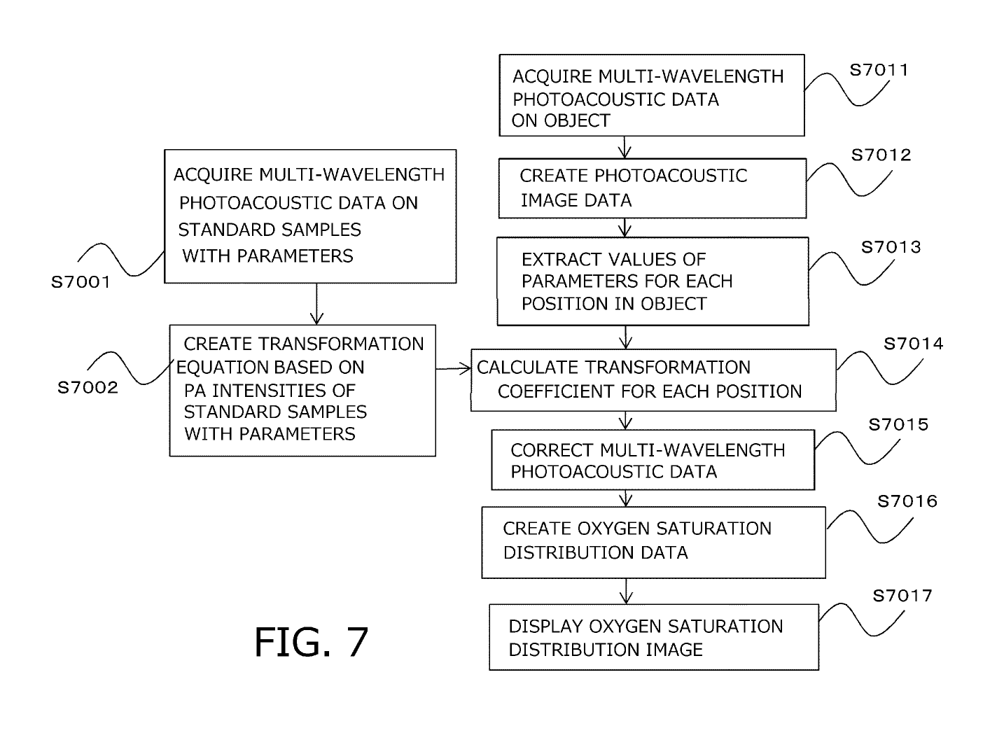

Now, a process flow in which the signal processing unit 612 determines the oxygen saturation distribution will be described using FIG. 7. Steps in S7011 to S7013 are similar to steps in S5011 to S5013 in Embodiment 2 and will not be described below.

In step S7014, the photoacoustic-wave data correcting unit 618 acquires the transformation coefficient for the photoacoustic signal and the absorbance (or the absorption coefficient) at each wavelength for each position, based on the transformation equation stored in the storage apparatus 619 and the value of each parameter for the light absorber 110 for the position extracted in S7013.

Each of the transformation equations stored in the storage apparatus 619 has the measurement wavelength as a variable. The transformation coefficient may have, as a variable, at least one of the parameters such as the thickness, angle, and depth of the light absorber 110. That is, the transformation equation in the present embodiment is a relational expression that represents the relation between the wavelength of light and the feature amount such as the thickness, angle, or depth of the light absorber 110 and the transformation coefficient.

When the object 111 is measured, the transformation equations are preferably pre-created and stored in the storage apparatus. However, the transformation equations may be created after the object 111 is measured.

Data used to derive the transformation equations is acquired by measuring a standard sample with a known absorption spectrum using the photoacoustic apparatus in the present embodiment. In this case, a standard sample produced as in the case of S5014 is measured at the wavelengths used to measure the object 111. Then, the parameter of interest is determined, and the transformation coefficient is calculated. At this time, measurement results with specific values are used for parameters not of interest. For example, when the parameter is the depth d, α^{λ}^{ᵢ}[d] is calculated as follows.

[Math. 9]

$${α}^{{λ}_{i}} [ d ] = \frac{{C}^{{λ}_{i}}}{{PA}^{{λ}_{i}} [ d ]} \qquad ( 9 )$$

A coefficient that represents the absorption of light at each wavelength by the standard sample is denoted by C^{λ}^{ᵢ}. For example, the absorbance or the absorption coefficient may be utilized as the coefficient. A photoacoustic signal measured at each depth is denoted by PA^{λ}^{ᵢ}[d], and as the photoacoustic signal, a reception signal may be used which is output from the photoacoustic-signal collecting unit 617 when the standard sample is measured at each wavelength. Examples of expression of the intensity of the reception signal include the maximum value for the photoacoustic signal (PEAK value), the difference between the maximum value and the minimum value for the photoacoustic signal (PEAK TO PEAK value), and the maximum value or the integration value for the photoacoustic wave resulting from envelope detection.

Then, based on the resultant transformation coefficients α^{λ}^{ᵢ}[d] for various depths, transformation equations α^{λ}^{ᵢ}(d) having the depth as a variable are derived using the method of least squares and the like. Any parameter other than the depth d (for example, the inclination or the thickness) may be used for the transformation equations. Alternatively, two or more parameters may be used.

For the standard sample, samples with various thicknesses, angles, and shapes are prepared, and each of the samples is measured at each wavelength. The samples are further measured with the depth thereof varied. Measuring various samples as described above allows acquisition of transformation coefficients corresponding to various values of each parameter. The material for the standard sample or the like is similar to the material in Embodiment 1 and will not be described below.

When, for example, the measurement wavelengths for the object 111 are not predetermined, transformation equations having the wavelength λ as a variable may be created. In this case, the standard sample is measured at various wavelengths in wavelength regions near the wavelengths at which measurement may be made.

The resultant transformation equations are stored in the photoacoustic-wave data correcting unit or the memory. Then, the value of the parameter for the light absorber 110 for each position extracted in S7013 is substituted into the transformation equation to allow the transformation coefficient for the photoacoustic signal and the absorbance (or the absorption coefficient) at each wavelength to be acquired for the position.

Steps S7015 to S7017 are similar to steps S5015 to S5017 in Embodiment 2 and will not be described below.

The present embodiment is applicable to apparatus configurations other than the apparatus configuration in FIG. 6. For example, the present embodiment is applicable to any light- or sound-focus photoacoustic microscopes and to any photoacoustic apparatuses that perform image reconstruction.

The use of the photoacoustic apparatus disclosed in the embodiment as described above allows correction of the effects of the beam profile and the amount of light at each wavelength radiated to the object, enabling specific-value information such as the oxygen saturation to be easily and accurately obtained.

In the above description, the transformation coefficient is calculated based on the intensity of the reception signal derived from the sample with known optical characteristics measured at each wavelength. To allow the oxygen saturation of the object to be determined, the concentration ratio between oxyhemoglobin and deoxyhemoglobin is determined. When this is applied to the present invention, first, the intensity of the reception signal is determined for each set of two wavelengths at which the respective components exhibit characteristic optical characteristics. Subsequently, each of the resultant intensities is converted into a specific value, and then, the oxygen saturation is determined. However, combinations of wavelengths suitable for determining the oxygen saturation are limited to some degree. Specifically, the combination includes a wavelength close to and shorter than 800 nm at which the absorption coefficients for the two components change to each other and a wavelength close to and longer than 800 nm, and a suitable combination of wavelengths is, for example, a combination of 700 nm and 850 nm. Therefore, new transformation coefficients can be calculated such that, with the two wavelengths fixed, the oxygen saturation is directly determined from the reception signal intensities at the respective wavelengths based on the transformation coefficients for the respective wavelengths.

The present invention can be implemented by a computer (or a device such as a CPU or an MPU) in a system or an apparatus that provides the functions of the above-described embodiments by loading and executing a program recorded in the storage apparatus. Alternatively, the present invention can be implemented by a method including steps executed by the computer in the system or apparatus that provides the functions of the above-described embodiments by loading and executing the program recorded in the storage apparatus. To accomplish this objective, the program is provided to the computer, for example, through a network or a recording medium of any of various types that can serve as the storage apparatus (that is, a computer-readable recording medium that holds data in a non-transitory manner). Therefore, the scope of the present invention includes all of the computer (including a device such as a CPU or an MPU), the method, the program (including a program code or a program product), and the computer-readable recording medium that holds the program in a non-transitory manner.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-127704, filed on Jun. 25, 2015, which is hereby incorporated by reference herein in its entirety.

What is claimed is:

1. An object information acquiring apparatus comprising:
a transformation coefficient acquiring unit configured to acquire, based on a lookup table or a relational expression that represents a relation between (a) a transformation coefficient for transformation between an intensity of an acoustic wave resulting from radiation of light to a standard sample with a known coefficient relating to light absorption and the coefficient relating to the light absorption of the standard sample and (b) a wavelength of light, the transformation coefficient corresponding to the wavelength of the light; and
a signal processing unit configured to acquire specific information on an object using the transformation coefficient and an electric signal derived from the acoustic wave generated by the object irradiated with light.

2. The object information acquiring apparatus according to claim 1, wherein the signal processing unit is configured to acquire an absorption coefficient of the object with respect to the wavelength of the light radiated to the object, using the transformation coefficient.

3. The object information acquiring apparatus according to claim 1, wherein
the signal processing unit is configured to acquire a plurality of electric signals derived from light with respective plurality of wavelengths,
the transformation coefficient acquiring unit is configured to acquire a plurality of transformation coefficients corresponding to the plurality of wavelengths, based on the lookup table or the relational expression, and
the signal processing unit is configured to acquire information on a concentration of a substance in the object as the specific information, using the plurality of electric signals and the plurality of transformation coefficients.

4. The object information acquiring apparatus according to claim 3, wherein the signal processing unit is configured to acquire an oxygen saturation as the concentration of the substance in the object.

5. The object information acquiring apparatus according to claim 1, wherein the coefficient relating to the light absorption is an absorbance or an absorption coefficient.

6. The object information acquiring apparatus according to claim 1, further comprising:
a feature amount acquiring unit configured to acquire a feature amount relating to at least one of a depth, a thickness, and an inclination of an absorber inside the object; and
a memory configured to store the lookup table or the relational expression representing the relation among the transformation coefficient and the wavelength of light and the feature amount, and
the transformation coefficient acquiring unit is configured to acquire the transformation coefficient, based on the lookup table or the relational expression stored in the memory and the feature amount acquired by the feature amount acquiring unit.

7. The object information acquiring apparatus according to claim 1, further comprising:
a light source;
a conversion element configured to receive an acoustic wave generated by the object irradiated with light from the light source, and convert the acoustic wave into an electric signal; and
a memory configured to store the lookup table or the relational expression.

8. The object information acquiring apparatus according to claim 1, further comprising an optical lens configured to focus light radiated from the light source.

9. The object information acquiring apparatus according to claim 8, wherein the optical lens is configured to focus light radiated from the light source as a light spot of 10 μm or less in size.

10. The object information acquiring apparatus according to claim 9, wherein the optical lens is configured to focus light radiated from the light source as a light spot of 1 μm or less in size.

11. The object information acquiring apparatus according to claim 1, further comprising an acoustic lens configured to focus the acoustic wave generated by the object.

12. The object information acquiring apparatus according to claim 1, further comprising an image display unit configured to display the specific information acquired by the signal processing unit.

13. The object information acquiring apparatus according to claim 7, wherein
the memory is configured to store the lookup table or the relational expression representing the relation between the transformation coefficient for each position in the object and the wavelength of light,
the transformation coefficient acquiring unit is configured to acquire the transformation coefficient for each position in the object corresponding to the wavelength of light, based on the lookup table or the relational expression stored in the memory, and
the signal processing unit is configured to acquire the specific information for each position in the object, using the electric signal and the transformation coefficient for each position in the object.

14. An object information acquiring method comprising:
acquiring a lookup table or a relational expression that represents a relation between (a) a transformation coefficient for transformation between an intensity of an acoustic wave resulting from radiation of light to a standard sample with a known coefficient relating to light absorption and the coefficient relating to the light absorption of the standard sample and (b) a wavelength of light;
acquiring the transformation coefficient corresponding to the wavelength of the light, based on the lookup table or the relational expression; and
acquiring specific information on an object, using the transformation coefficient and an electric signal derived from the acoustic wave generated by the object irradiated with light.

15. A non-transitory computer readable storage medium having a program stored therein, the program allowing a computer to execute:
acquiring a lookup table or a relational expression that represents a relation between (a) a transformation coefficient for transformation between an intensity of an acoustic wave resulting from radiation of light to a standard sample with a known coefficient relating to light absorption and the coefficient relating to the light absorption of the standard sample and (b) a wavelength of light;
acquiring the transformation coefficient corresponding to the wavelength of the light, based on the lookup table or the relational expression; and
acquiring specific information on an object, using the transformation coefficient and an electric signal derived from the acoustic wave generated by the object irradiated with light.
